(19) United States
(12) Patent Application Publication
(10) Pub. No.: US 2024/0100308 A1
(43) Pub. Date: Mar. 28, 2024

(54) CATHETER SYSTEM WITH HYDRAULICALLY INFLATABLE STABILIZERS

(71) Applicant: BIOTRONIK AG, Buelach (CH)

(72) Inventors: Marc Gianotti, Wiesendangen (CH); Dragana Margeta, Stetten (CH); Johannes Kulicke, Dachsen (CH); Michael Jetter, Thayngen (CH); Ulf Fritz, Bargen (CH)

(21) Appl. No.: 18/262,285

(22) PCT Filed: Jan. 28, 2022
(86) PCT No.: PCT/EP2022/051980

§ 371 Date: Jul. 20, 2023

Related U.S. Application Data
(60) Provisional application No. 63/143,984, filed on Feb. 1, 2021.

(30) Foreign Application Priority Data
Mar. 11, 2021 (WO) PCT/EP2021/056214

(51) Int. Cl. A61M 25/10; A61M 25/00

(52) U.S. Cl. CPC A61M 25/104; A61M 25/005; A61M 2025/0006; A61M 2025/1047; A61M 2025/1056; A61M 2205/0216

(57) ABSTRACT

PTA catheters and improved PTA methods address problems associated with PTA procedures. One or more stabilizers are activated to hold or fix the PTA catheter at a desired relative position within a support catheter. Timely activation of the stabilizers can prevent deformation of the PTA apparatus during manipulation of the PTA apparatus within biological vessels.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2022/051980, which was filed Jan. 8, 2022, which application claimed priority from U.S. Provisional Application Ser. No. 63/143,984, which was filed Feb. 1, 2021 and from PCT/EP2021/056214, which was filed Mar. 11, 2021.

### FIELD OF THE INVENTION

Fields of the invention catheter systems that are configured for the vessel of a patient and percutaneous transluminal angioplasty apparatuses.

### BACKGROUND

Percutaneous transluminal angioplasty (“PTA”) refers to methods for widening narrowed blood vessels using balloon catheters. In general, the blood vessels are narrowed by the presence of atherosclerotic plaques that form in the walls of the blood vessels. Atherosclerotic plaques contain lipids, inflammatory cells, smooth muscle cells, and connective-tissue cells and often form in areas of blood-vessel walls exposed to non-laminar or turbulent blood flow, including areas of blood-vessel walls near arterial branch points. The plaques often begin as early atherosclerotic lesions, referred to as “fatty streaks,” containing macrophage-induced lipid-laden foam cells formed by uptake of enzymatically oxidized lipids, including oxysterols and 4-hydroxynonenal, from circulating low-density lipoproteins and very-low-density lipoproteins. Macrophages secrete pro-inflammatory cytokines that recruit smooth-muscle cells to the lesion and that stimulate growth of additional macrophages, resulting in growth and development of the early atherosclerotic lesions into subendothelial fibrous plaques with fibrous caps that may, in turn, become calcified. Atherosclerotic lesions are complex matrixes of various types of cells, cell remnants, lipids, oxidized lipids, inorganic ions, and even invasive bacteria. Atherosclerotic lesions lead to constrained blood flow, resulting in ischaemic conditions that are represented by insufficient perfusion of blood to vessels, limbs, and vital organs, often accompanied by the formation of blood clots and further lesion growth which, in turn, are referred to as “stenosis,” and may lead to acute life-threatening conditions, such as myocardial infarction, or chronic limb ischemia.

In percutaneous transluminal angioplasty (“PTA”) methods, a pre-folded and deflated balloon catheter, referred to as a “PTA catheter,” is inserted into a blood vessel over a previously inserted guidewire and the deflated balloon is moved over the guidewire into a narrowed portion of the blood vessel while the position of the deflated balloon is monitored by X-ray fluoroscopy or magnetic-resonance-imaging (“MRI”). A pressurized inflation fluid introduced into an inflation port at the proximal end of the PTA catheter then inflates the balloon, which, in turn, expands the blood vessel and disrupts the atherosclerotic lesion. Application of PTA methods may result in complications. Over-inflation of the balloon can result in undesirable persistent distention of a blood vessel which, in turn, may disrupt laminar blood flow within and near the distention and lead to new atherosclerotic lesions or regrowth of the treated atherosclerotic lesion. Localized forces produced by balloon inflation can also induce fissures and tears in the inner blood-vessel-wall lining that result in blood flow into a false lumen, or channel, between blood-vessel-wall, referred to as “dissection.” In more serious cases, these localized forces may result in a rupture, dissection, hematoma or pseudoaneurysm.

The use of drug-eluting PTA catheters may decrease the risks of certain types of PTA complications, but complications due to over-inflation and undesirably large local forces produced during balloon inflation remain a significant problem associated with currently practiced PTA methods. For enhanced maneuvering and vessel shielding purposes, PTA catheters can be used within support catheters, the entire apparatus, referred to as a “PTA apparatus,” including a guidewire, a PTA catheter, and a support catheter, along with additional components and features. The PTA apparatus or system can be thought of as a first flexible tube, slidably mounted over a guidewire, within a second flexible tube. During use, the first and second tubes move relative to one another while the entire apparatus is manipulated within generally curved biological vessels. These movements may be accompanied with various forces and deformations, which can exacerbate the risks of apparatus failures and greatly complicate PTA-catheter-based manipulation and procedures. For this reason, designers, developers, and vendors of PTA instrumentation as well as PTA practitioners continue to seek improved PTA catheters, improved PTA apparatuses, and improved PTA methods that decrease or eliminate the risks of various types of PTA complications.

### SUMMARY OF THE INVENTION

A catheter system includes or consists of a first catheter having a first lumen surrounded by a first catheter shaft, and a second catheter that is inserted into the first lumen of the first catheter. The second catheter includes a second lumen surrounded by a second catheter shaft and one or more hydraulically inflatable stabilizers being in fluid communication with the second lumen. The one or more stabilizers is/are adapted when being hydraulically inflated via the second lumen to form an inflation-fluid chamber being inflatable radially outward to compress the stabilizer against an inner surface of the first catheter shaft and fix the position of the second catheter within the first catheter and constrain the second catheter to a coaxial disposition with respect to the first catheter.

### BRIEF DESCRIPTION OF THE DRAWINGS

Embodiments of the invention shall be described in detail with reference to the drawings. It is to be noted that the embodiments are not limiting for the invention, but merely represent illustrative examples.

FIG. 1A-B illustrate an atherosclerotic lesion,

FIG. 2 illustrates a PTA catheter,

FIG. 3A-G illustrate inflation of the balloon of a PTA catheter,

FIG. 4A-B illustrates one class of implementations of the currently disclosed improved PTA catheter and improved PTA apparatus,

FIG. 5 illustrates a hydraulically activated stabilizer in a cross-section view of a portion of an improved PTA apparatus,

FIG. 6 shows a multi-stabilizer implementation of an improved PTA apparatus in cross-section,

FIG. 7A-C illustrate certain problems that arise when a PTA apparatus is manipulated during a PTA procedure,

FIG. 8 illustrates another benefit of stabilizers within an improved PTA apparatus.

### DETAILED DESCRIPTION OF THE PREFERRED EMBODIMENTS

The present invention is related to catheter system including or consisting of a first catheter having a first lumen surrounded by a first catheter shaft; and a second catheter that is inserted into the first lumen of the first catheter, and the second catheter having a second lumen surrounded by a second catheter shaft and one or more hydraulically inflatable stabilizers being in fluid communication with the second lumen. The one or more stabilizers is/are adapted when being hydraulically inflated via the second lumen to form an inflation-fluid chamber being inflatable radially outward thereby compressing the stabilizer against an inner surface of the first catheter shaft and fixing the position of the second catheter within the first catheter and constrain the second catheter to a coaxial disposition with respect to the first catheter.

The application is further directed to an improved percutaneous transluminal angioplasty apparatus, used to treat obstructed arteries, veins, and other vessels within biological tissues, that includes stabilizers that are activated to fix the relative positions of components of the percutaneous transluminal angioplasty apparatus, which are translated with respect to one another during certain portions of percutaneous transluminal angioplasty procedures, to prevent relative motion of the components during other portions of the percutaneous transluminal angioplasty procedures.

The current document is directed to improved PTA catheters, improved PTA apparatuses, and improved PTA methods that address problems associated with PTA procedures. Implementations of the improved PTA catheters include PTA catheters with one or more stabilizers that are activated to hold or fix the PTA catheter at a desired relative position within a support catheter. The improved PTA methods employ timely activation of the stabilizers to prevent deformation of the PTA apparatus during manipulation of the PTA apparatus within biological vessels. Certain implementations include hydraulically activated stabilizers, while other implementations employ one or more stabilizers activated by activation methods other than hydraulic activation.

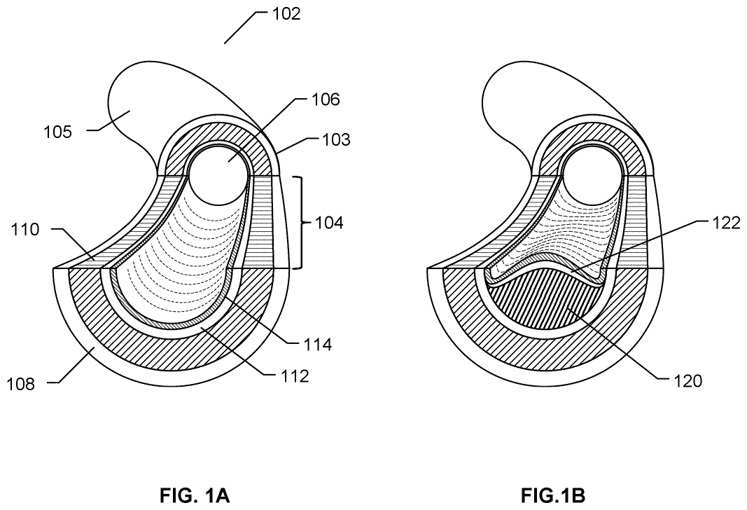

FIGS. 1A-B illustrate an atherosclerotic lesion. Figure TA illustrates a portion of a healthy, normal artery. The artery 102 is shown in a cutaway diagram, partly transected 103 and then lengthwise dissected 104. The artery 102 includes an artery wall 105 and an interior lumen 106 through which blood flows. The artery wall includes three main layers: (1) the adventitia 108, including several layers of connective tissue that anchor the artery to adjacent, external tissue; (2) the media 110, including smooth-muscle cells; and (3) the intima 112, including several layers that include an innermost endothelium 114 that forms the inner surface of the artery wall. FIG. 1B illustrates an atherosclerotic lesion. The atherosclerotic lesion 120 occupies a subendothelial volume between the endothelium and internal layers of the intima. As discussed above, the atherosclerotic lesion grows from an initial fatty streak to a complex matrix of various types of cells, cell remnants, lipids, oxidized lipids, inorganic ions, and even invasive bacteria, often capped by a fibrous layer 122 that may become calcified, over time. As also discussed above, PTA methods are used to disrupt the atherosclerotic lesion and reopen the partially or fully occluded arterial lumen.

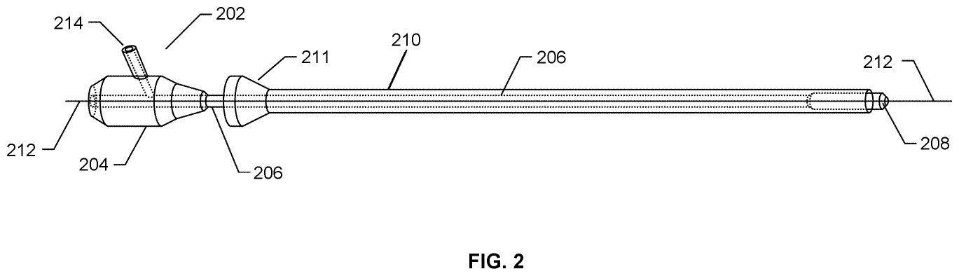

FIG. 2 illustrates a PTA catheter. PTA catheters are representative of a more general family of inflatable-member instruments used in general percutaneous-transluminal-interventional methods. The PTA catheter 202 includes a manifold 204, an outer shaft 206, and a deflated balloon 208 at the distal end of the shaft. The shaft of the PTA catheter is shown inserted into the lumen of a support catheter 210 via a manifold 211 located at the proximal end and over a guidewire 212. The balloon is inserted into a support catheter when a length-adjustable-balloon configuration is desired or, in certain cases, when additional guidance or shielding of the vessel is desired. Transradial artery access or transfemoral artery access is used to introduce an introducer sheath into the vascular system. The guidewire is inserted through the introducer sheath and navigated through the vasculature to and past an atherosclerotic lesion to be treated. The support catheter 210 is inserted over the guidewire and navigated to a desired position with respect to the lesion. The PTA catheter is mounted over the guidewire and inserted into the support catheter in order to navigate the balloon to a proper position with respect to the lesion, after which the balloon is inflated by introducing a pressurized liquid, formed from contrast agent and saline solution, into an inflation port 214 that extends from the manifold and that communicates through an outer PTA-catheter lumen to the interior of the balloon.

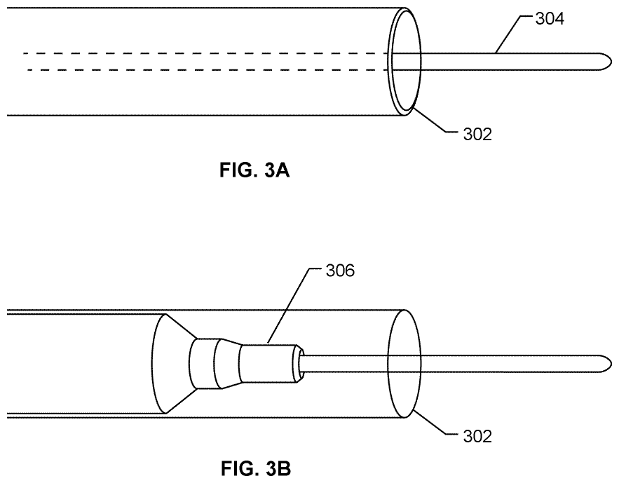

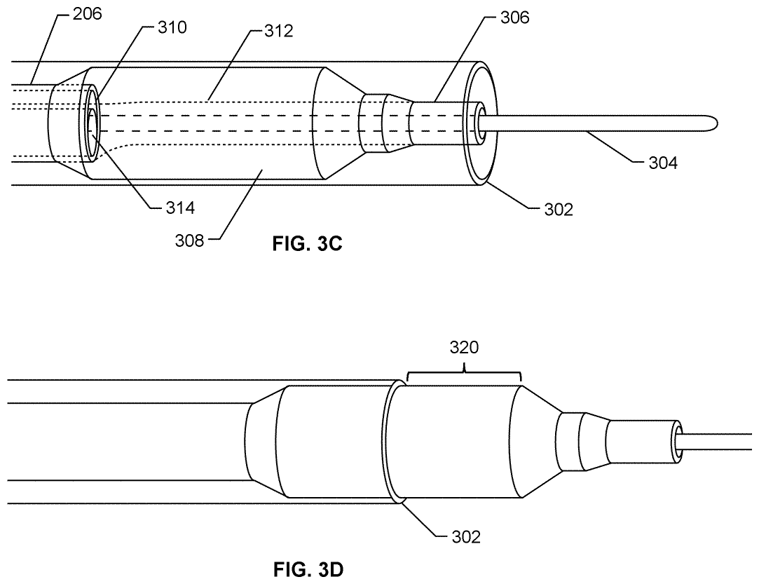

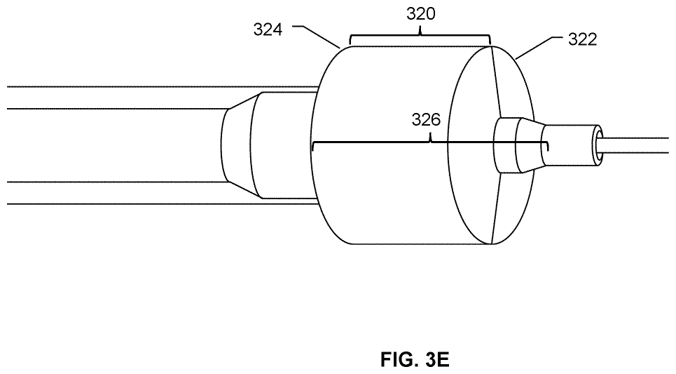

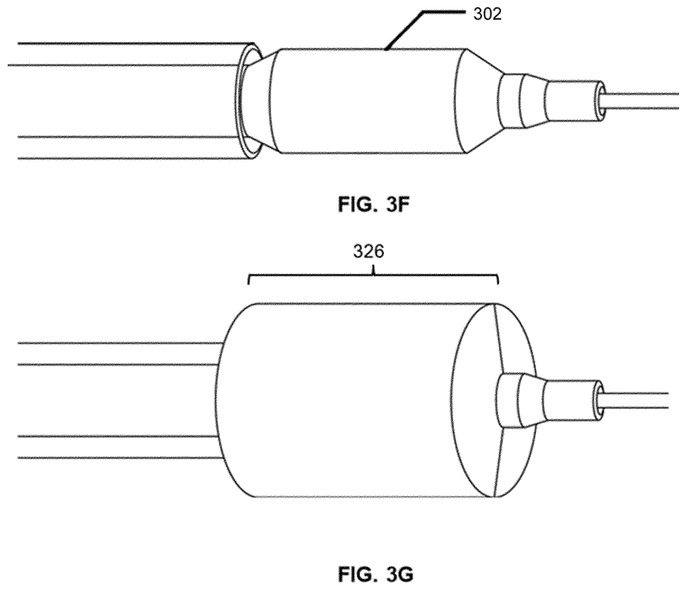

FIGS. 3A-G illustrate inflation of the balloon of a PTA catheter. A balloon-length-adjustable PTA catheter is used as an example of a PTA catheter, in FIGS. 3A-G. FIG. 3A illustrates the open, distal end of a support catheter 302 through which a guidewire 304 passes. The distal end of the support catheter is navigated to an appropriate position relative to the lesion to be treated. As shown in FIG. 3B, the distal end of the shaft of a PTA catheter 306 is advanced along the guidewire towards the distal end 302 of the support catheter. As shown in FIG. 3C, the distal end of the PTA catheter 306 is advanced further along the guidewire to a point that where it is flush with the distal end 302 of the support catheter. The deflated balloon 308 is fully visible in this figure. The inflation fluid is introduced into the inflation port (214 in FIG. 2) and flows under pressure through a PTA-catheter inflation lumen 310 within the PTA-catheter shaft 206 that is in open communication with the balloon 308. The PTA-catheter shaft 206 further extends into a distal shaft portion 312. The guidewire 304 passes through an inner guide-wire lumen 314 within the PTA-catheter that is not in fluid communication with the PTA-catheter inflation lumen. In certain implementations, the inflation lumen may be coaxially arranged around the guide wire lumen. The lumen cross-sections may be cylindrical or, in certain implementations, may have more complex shapes.

As shown in FIG. 3D, the PTA catheter is further advanced, along the guidewire, so that a desired length, or portion, of the balloon 320 is exposed, past the distal end 302 of the support catheter, to the interior environment of the blood vessel. The inflation fluid is then introduced into the PTA-catheter inflation lumen and pressurized in order to inflate the portion of the balloon exposed to the interior environment of the blood vessel, as shown in FIG. 3E. In this depiction, the inflated balloon appears cylindrical, with relatively sharp edges 322 and 324 at the balloon shoulders, to emphasize the length of the inflated portion of the balloon. However, in real-world instruments, the inflated portion of the balloon has a rounded shape. In FIG. 3F, the deflated balloon is further advanced along the guidewire, prior to inflation, so that the full length of the balloon 320 can be subsequently inflated, as shown in FIG. 3G. Thus, the length 326 of the inflated a balloon can be adjusted by positioning of the distal end of the PTA catheter shaft relative to the distal end of the support catheter. In conventional PTA instrumentation, the balloon is fully inflated in the absence of support catheters as constraining members, and balloon-length adjustment is achieved by selecting, prior to deployment during treatment, one or more PTA catheters, each having a balloon of a different, but constant, length.

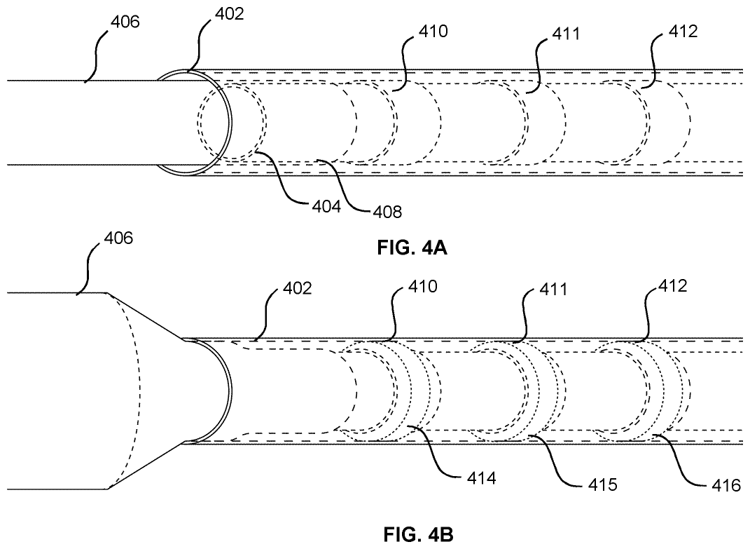

FIGS. 4A-B illustrates one class of implementations of the currently disclosed improved PTA catheter and improved PTA apparatus. In FIG. 4A, a portion of the improved PTA apparatus is shown. The illustrated portion of the PTA apparatus includes the distal end of the support catheter 402, the distal end of the PTA catheter 404, which is recessed within the support catheter, and a portion of the balloon 406 which is shown protruding outward from the support catheter and mounted to the PTA catheter 408 on a distal end segment of the PTA catheter. In addition, three not yet inactivated stabilizers 410-412 are shown at positions along the PTA catheter to the right of the portion of the balloon 408 mounted to the distal end segment of the PTA catheter. In FIG. 4A, the balloon is uninflated or minimally inflated.

FIG. 4B shows the PTA apparatus, previously shown in FIG. 4A, following inflation of the balloon via introduction of pressurized inflation fluid into the inflation lumen of the PTA catheter, as discussed above. In this case, the balloon 406 has expanded radially, as have the stabilizers 410-412. Radial expansion of the stabilizers is constrained by the support catheter 402. However, radial expansion of the stabilizers proceeds, as the balloon is inflated, to tightly compress portions of the expanding stabilizers against the support catheter to form annular cylindrical bands 414-416. The pressure of the inflation fluid therefore results in a series of compressed annular sections, -bands or O-ring-like ribs, along the PTA catheter that both fix the translational position of the PTA catheter relative to the support catheter as well as fix the position and orientation of the PTA-catheter shaft within the support catheter so that the long axes of apparent symmetry of the PTA catheter and support catheter are coextensive, or, in other words, so that the PTA catheter and support catheter are constrained by the stabilizers to remain coaxial. In this implementation, the stabilizers are hydraulically activated by the same pressurized inflation fluid that hydraulically activates the balloon. Various implementations use between one and three, four, or more stabilizers. In the implementation discussed with reference to FIGS. 4A-B, the stabilizers are hydraulically activated along with the balloon. However, in alternate implementations, stabilizers may be separate activated by other means, including by additional mechanical stabilizer-activating components that may involve changing the relative rotational orientations of the PTA catheter and support catheter to, for example, rotate cam-like stabilizers into locking positions, or manipulating one or more additional mechanical-activation components, such as an internal stabilizer-activation tubular sheath.

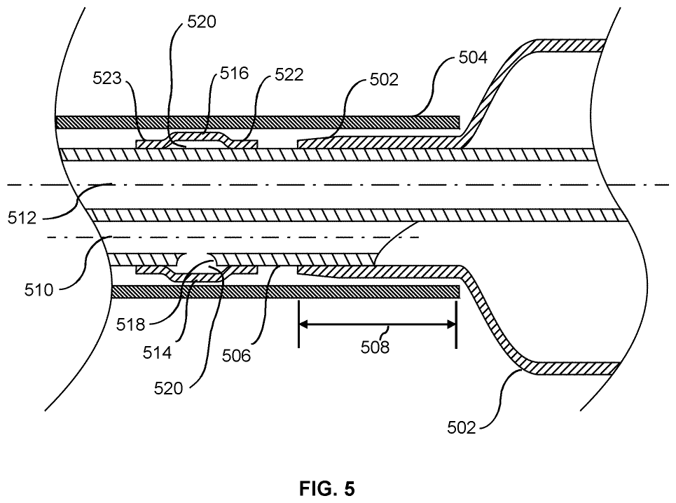

FIG. 5 illustrates a hydraulically activated stabilizer in a cross-section view of a portion of an improved PTA apparatus. The illustrated portion of the improved PTA apparatus includes a portion of the balloon 502, support catheter 504, and PTA catheter 506. A proximal portion of the balloon of length 508 is recessed within the support catheter. Much of this recessed portion of the balloon is affixed to the outer surface of a distal end segment of the PTA catheter. The balloon may be affixed to the PTA catheter by thermal, ultrasound or laser welding, adhesives, mechanical tension, or by other means. In many implementations, a recessed portion of the balloon must be maintained to preserve a folding of the balloon so that the balloon can be deflated and retracted into the support catheter prior to retracting the PTA apparatus from the vessel or moving the PTA apparatus within the vessel. As discussed above, the PTA catheter includes an inflation lumen 510 and a guide-wire lumen 512. The stabilizer in this implementation is, as discussed above, an annular band that, in cross-section, appears as the annular-band sections 514 and 516. At least one port 518 in the PTA-catheter shaft allows inflation fluid to pass from the inflation lumen into an annular chamber 520 formed between a central, expanded portion of the annular band and the outer surface of the PTA-catheter shaft. As discussed above, with reference to FIG. 4B, when pressurized inflation fluid is introduced into the inflation lumen, the annular chamber expands and compresses the outer surface of a portion of the annular band against the inner surface of the support catheter, forming a compressed annular section, band, or O-ring-like rib that both fixes the translational position of the PTA catheter with respect to the support catheter and also fixes the PTA catheter to be disposed within the support catheter in a coaxial position with respect to the support catheter.

In the illustrated implementation, the stabilizer 514 and 516 is a separate annular band of material from the balloon. In certain alternative implementations, the stabilizer may be a portion of the balloon material that is not bonded to the underlying PTA-catheter shaft and that overlies one or more ports in the PTA-catheter shaft so that, when inflation fluid is pressurized within the inflation lumen of the PTA catheter, an annular bulge is pushed radially outward and against the inner surface of the support catheter. For each stabilizer, there may be one, two, or more ports arranged along a circular band of the PTA-catheter shaft underlying the portion of the stabilizer that is expanded outward by pressurized inflation fluid. In certain implementations, the stabilizer material may have a different elasticity, deformability, or compliance than the balloon material. The difference in elasticity, deformability, or compliance between the two different types of material can be selected in order that compression of the stabilizer against the inner surface of the support-catheter shaft occurs at various predetermined points in time relative to a particular degree of balloon expansion. In other implementations, the stabilizer material may have a different coefficient of friction with respect to the balloon and/or support-catheter material. Thus, depending on the selected materials, the translational position of the PTA catheter may be fixed soon after introduction of pressurized inflation fluid into the inflation lumen and prior to expansion of the balloon, may be instead fixed at a point in time coincident with a degree of partial balloon expansion, or may be fixed at a point in time coincident with, or following, full balloon expansion. The elasticity, deformability, compliance, or friction of the stabilizer material may vary even within the stabilizer, with the central portion of the stabilizer band more flexible and having greater elasticity so that the central portion of the stabilizer band preferentially expands radially with respect to the outer portions 522 and 523 of the band. In the illustrated implementation, stabilizer material that is expanded to form annular stabilizer bands is not affixed by adhesives or welding to the PTA catheter shaft, while the portions 522 and 523 at the edges of the stabilizers are fixed to the PTA catheter shaft by adhesives, welding, mechanical tension, or by other means. The stabilizer material can be selected from a range of materials, including polymers, fibers and metals, including elastomeric and/or duroplastic polymers, ductile, spring- and/or -shape-memory metals, and combinations of such materials.

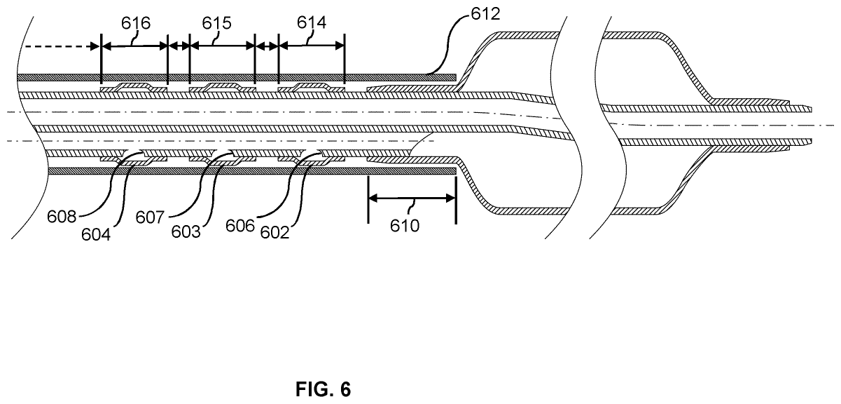

FIG. 6 shows a multi-stabilizer implementation of an improved PTA apparatus in cross-section. In this implementation, there are at least three stabilizers 602-604 that are inflated by inflation fluid passing through at least three corresponding ports 606-608 in the PTA-catheter shaft. As with the implementation shown in FIG. 5, a proximal portion of the balloon of length 610 is recessed within the support catheter 612, and much of this portion of the balloon is affixed to the distal end of the PTA catheter. In the implementation shown in FIG. 6, the widths 614-616 of the annular stabilizer bands are equal to one another, but, in alternative implementations, the widths and separation distances of the bands may vary. In addition, the elasticity, deformability, compliance, and/or coefficient of friction of the stabilizer material may also vary, to provide a progressive activation of stabilizers during inflation of the balloon.

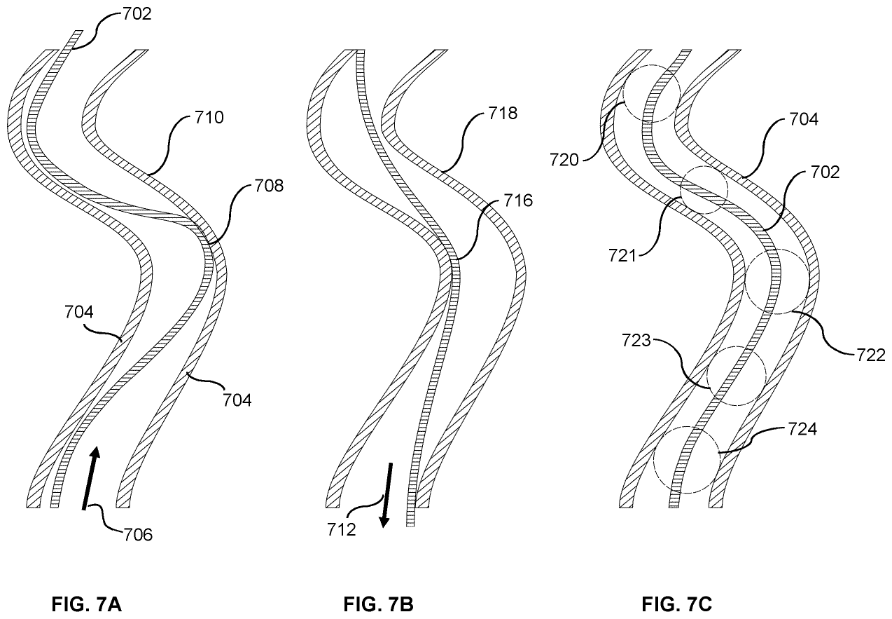

FIGS. 7A-C illustrate certain problems that arise when a PTA apparatus is manipulated during a PTA procedure. In FIG. 7A, a first inner tubular element 702, within a second outer tubular element 704, is being pushed upward relative to the outer tubular element, as indicated by arrow 706. Due to the different flexibilities and dimensions of the two tubular elements, and due to the curvature of the outer tubular element, the upward force exerted on the inner tubular element can result in bending or kinking of the inner tubular element with respect to the outer tubular element. This can result in significant forces applied by the inner tubular element to the outer tubular element at certain positions related to kinks, such as at kink 708, and wide separations of the inner tubular element from the outer tubular element at other positions, such as position 710. These widely varying forces result in frictional forces that require application of excessive forces in order to continue to move the inner tubular element upward relative to the outer tubular element, stored mechanical energy, and uneven translation of the inner tubular element with respect to the outer tubular element under application of a constant upward force, and a variety of derivative forces that can lead to local distortions in the shapes of the two tubular elements. Similarly, as shown in FIG. 7B, when the inner tubular element is retracted via a downward force, as indicated by arrow 712, much greater forces may end up applied by the inner tubular element to the outer tubular element at certain locations, such as location 716, with respect to other locations, such as location 718. These differing applied forces at different locations can again lead to local distortions in the shapes of one or both of the tubular elements, the sudden and undesired release of stored mechanical energy, and uneven translation of the inner tubular element with respect to the outer tubular element under application of a constant, downward applied force to the inner tubular element. FIG. 7C illustrates the beneficial effects provided by the above-mentioned stabilizers. In FIG. 7C, circles 720-724 represent activated stabilizers, which constrain the inner tubular element 702 to remain generally coaxially positioned with respect to the outer tubular element 704. As a result, many of the kinks, bands, and distortions discussed with reference to FIG. 7 A-B are prevented. Smooth translation of the PTA catheter, analogous to the inner tubular element of FIGS. 7A-C, with respect to the outer tubular member, analogous to the outer tubular element of FIGS. 7A-C, is critical during PTA procedures. Excessive applied forces, stored mechanical energy, and other such problems, can lead to the many different deleterious effects and results discussed above, including apparatus failures, lack of accuracy, difficulty in maneuvering, device mispositioning, and vessel damage. Thus, stabilizers, when fully activated, apply sufficient radial forces to fix the relative translational positions of the PTA catheter and support catheter, and when partially activated, such as when the balloon is partially inflated, maintain a relative coaxial disposition of the PTA catheter within the support catheter, to facilitate smooth translation of the PTA catheter with respect to the support catheter and to prevent distortions of the PTA apparatus during movement within vessels, particularly curved vessels. Further, the stabilizers, when partially or fully engaged with the support catheter, reduce or prevent strain, or forces, that are otherwise exerted, during push or pull operations of the PTA catheter, onto both support catheter and surrounding vessel walls.

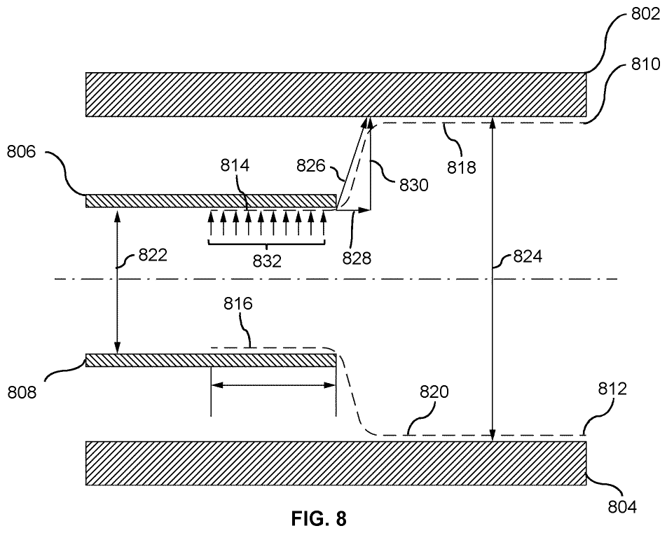

FIG. 8 illustrates another benefit of stabilizers within an improved PTA apparatus. FIG. 8 shows, in cross-section, a portion of a PTA apparatus similar to the portion shown in FIG. 5 within a biological vessel 802 and 804. A distal end section of the support catheter 806 and 808 is positioned within the vessel. When the balloon 810 and 812 is inflated to conform to the inner surface of the vessel 818 and 820 and apply forces to the vessel walls, the balloon is constrained within the distal end section of the support catheter 814 and 816 and then expands radially from a first diameter 822 within the support catheter to a second diameter 824 external to the support catheter. Expansion of the balloon results in a force 826 roughly parallel to an averaged linear portion of the curved surface of the balloon between the support catheter and the vessel that can be decomposed into a lateral, or axial, force 828 and a vertical, or radial force 830. Of course, the portion of the balloon between the support catheter and the vessel approximates a conical section, as a result of which the radial forces lie in an annular section of an imaginary planar disk perpendicular to the axis of symmetry of the support catheter and vessel, and the axial forces lie in an imaginary cylindrical continuation of the inner surface of the support catheter. During inflation, the axial and radial forces may place large stresses on the attachment of the balloon to the PTA catheter. In addition, the radial forces tend to fix the position of the PTA catheter relative to the support catheter while the axial forces tend to pull the PTA catheter out from the support catheter, resulting in a lengthening of the inflated portion of the balloon. One approach to stabilizing the PTA catheter within the support catheter during balloon inflation is to use wider recessed portions of the balloon along the distal portion of the PTA catheter in order to increase the radial force applied to the support catheter, indicated by the small radial arrows 832. However, use of the stabilizers, discussed above, can alternatively provide the additional needed radial forces to counteract the axial forces produced by balloon inflation. Stabilizers may also help distribute radial forces against the support catheter over a greater length of the support-catheter shaft to prevent distortion of the distal end of the support catheter by expansion forces generated by balloon expansion.

Although the present invention has been described in terms of particular embodiments, it is not intended that the invention be limited to these embodiments. Modifications in analogy to the embodiments presented will be apparent to those skilled in the art. For example, a variety of different materials can be used for hydraulically activated stabilizers, with varying elasticities and deformabilities. Multiple stabilizers may be created from a single wide band, or sleeve, of stabilizer material, as discussed above. Different numbers of circularly disposed ports may be used for inflation of a particular stabilizer.

Further examples of the present disclosure are provided below:
A. A percutaneous transluminal angioplasty (“PTA”) apparatus including:
   a guide wire;
   a support catheter; and
   a PTA catheter that is inserted into the support catheter, the PTA catheter having
      a distal end,
      a proximal end,
      an outer shaft
      a catheter balloon mounted to the distal end,
      an inflation lumen in fluid communication with the internal volume of the catheter balloon,
      a guide-wire lumen through which the guide wire passes, and
      one or more stabilizers, that, when activated, preferably via the inflation lumen, fix the position of the PTA catheter within the support catheter and constrain the PTA catheter to a coaxial disposition with respect to the support catheter.
B. The PTA apparatus of A wherein the support catheter has a tubular shaft of a first diameter that is open at the distal end and that opens to a manifold at the proximal end.
C. The PTA apparatus of B wherein the PTA catheter further includes:
   a manifold mounted to the proximal end of the outer shaft;
   an inflation port that provides fluid communication through the manifold to the inflation lumen; and
   a guide-wire port through which a guide wire is inserted into the guide-wire lumen.
D. The PTA apparatus of any of A-C wherein the one or more stabilizers each includes:
   a band of stabilizer elastomeric material mounted to the surface of the PTA-catheter shaft; and
   one or more inflation-fluid ports below a central portion of the band of stabilizer elastomeric material.
E. The PTA apparatus of D
   wherein a central portion of the band of stabilizer elastomeric material is not affixed to the outer surface of the PTA-catheter shaft;
   wherein two edge portions of the band of stabilizer elastomeric material are affixed to the outer surface of the PTA-catheter shaft, forming an expandable, annular inflation-fluid chamber between the central portion of the band of stabilizer elastomeric material and the underlying outer surface of the PTA-catheter shaft and one or more inflation-fluid ports.
F. The PTA apparatus of E wherein the inflation-fluid chamber is adapted to be inflated radially outward, compressing the stabilizer against the inner surface of the support-catheter shaft, by a pressurized inflation fluid, when the pressurized inflation fluid is introduced into the inflation lumen of the PTA catheter and the pressurized inflation fluid flows through the inflation-fluid ports into the annular inflation-fluid chamber.
G. The PTA apparatus of F wherein the band of stabilizer elastomeric material is separate from the PTA-catheter balloon and separate from the bands of stabilizer elastomeric material of other stabilizers.
H. The PTA apparatus of F or G wherein the band of stabilizer elastomeric material is a portion of band of a portion of a stabilizer elastomeric material that forms two or more adjacent stabilizers along the PTA-catheter shaft, each overlying one or more inflation-fluid ports in the PTA-catheter shaft.
I. The PTA apparatus of any of F-H wherein the PTA-catheter shaft includes two or more stabilizers, each including a band of stabilizer elastomeric material with an elasticity, deformability, and/or compliance different from that of the band or bands of the other stabilizers, so that the stabilizers are fully activated at different points in time as inflation fluid is introduced into the inflation lumen of the PTA catheter.
J. The PTA apparatus of any of F-I wherein the band of stabilizer elastomeric material has an elasticity, deformability and/or compliance different from that of the PTA-catheter balloon so that the stabilizer is fully inflated at a different point in time than the point of time when the PTA-catheter balloon is fully inflated.
K. The PTA apparatus of J wherein the stabilizer is adapted to be inflated prior to partial inflation of the PTA-catheter balloon.
L. The PTA apparatus of J wherein the stabilizer is adapted to be inflated when the PTA-catheter balloon is partially inflated.
M. The PTA apparatus of J wherein the stabilizer is adapted to be inflated when the PTA-catheter balloon is fully inflated.
N. The PTA apparatus of J wherein the stabilizer is adapted to be inflated after the PTA-catheter balloon is fully inflated.
O. The PTA apparatus of J wherein the stabilizer is adapted to be inflated together with a portion of the PTA-catheter balloon that is recessed within the support catheter, resulting in a combination of radial forces of the recessed PTA balloon portion and stabilizer, and applied to the support catheter, that counteract axial forces produced by balloon inflation, thereby fixing the position of the PTA catheter relative to the support catheter and constraining the PTA catheter to a coaxial disposition with respect to the support catheter.
P. The PTA apparatus of J wherein the stabilizer is adapted to be inflated together with the PTA-catheter balloon recessed within the support catheter, resulting in a combination of radial forces of the recessed PTA balloon and stabilizer, and applied to the support catheter, that fix the position of the PTA catheter relative to the support catheter, thereby reducing or preventing strain, or forces, that are otherwise exerted, during push or pull operations of the PTA catheter.
Q. A method that treats a lesion within a vessel lumen of a patient, the method including: introducing a guide wire into the vessel lumen;
   introducing a support catheter into the vessel lumen over the guide wire;
   introducing a PTA catheter with one or more stabilizers into the supporter catheter over the guide wire; and
   activating the one or more stabilizers to fix the position of the PTA catheter within the support catheter and constrain the PTA catheter to a coaxial disposition with respect to the support catheter.
R. The method Q
   wherein the support catheter has a tubular shaft of a first diameter that is open at the distal end and that opens to a manifold at the proximal end; and
   wherein the PTA catheter further includes:
   a manifold mounted to the proximal end of the outer shaft;
   an inflation port that provides fluid communication through the manifold to the inflation lumen; and
   a guide-wire port through which a guide wire is inserted into the guide-wire lumen.
S. The method of R wherein the one or more stabilizers each includes:
   a band of stabilizer elastomeric material mounted to the surface of the PTA-catheter shaft; and
   one or more inflation-fluid ports below a central portion of the band of stabilizer elastomeric material.
T. The method of S
   wherein a central portion of the band of stabilizer elastomeric material is not affixed to the outer surface of the PTA-catheter shaft;
   wherein two edge portions of the band of stabilizer elastomeric material are affixed to the outer surface of the PTA-catheter shaft, forming an expandable, annular inflation-fluid chamber between the central portion of the band of stabilizer elastomeric material and the underlying outer surface of the PTA-catheter shaft and one or more inflation-fluid ports.
U. The method of T wherein, when pressurized inflation fluid is introduced into the inflation lumen of the PTA catheter, pressurized inflation fluid flows through the inflation-fluid ports into the annular inflation-fluid chamber and inflates the inflation-fluid chamber radially outward, compressing the stabilizer against the inner surface of the support-catheter shaft.
V. The PTA apparatus of any of A-P for use in the treatment of a lesion within a vessel lumen of a patient.
W. A catheter system including or consisting of
   a first catheter having a first lumen surrounded by a first catheter shaft, (preferably) having a uniform circumference and/or diameter; and
   a second catheter that is inserted into the first lumen of the first catheter, and
   the second catheter having a second lumen surrounded by a second catheter shaft and one or more hydraulically inflatable stabilizers being in fluid communication with the second lumen, and wherein the one or more stabilizers is/are adapted when being hydraulically inflated via the second lumen to form an inflation-fluid chamber being inflatable radially outward thereby compressing the stabilizer against an inner surface of the first catheter shaft and fixing the position of the second catheter within the first catheter and constrain the second catheter to a coaxial disposition with respect to the first catheter.
X. The catheter system according to W, wherein the first catheter is a support catheter or a guiding catheter and/or the second catheter is a balloon catheter, another guiding catheter, a percutaneous transluminal angioplasty catheter, a dilator catheter, a crossing catheter or a microcatheter.
Y. The catheter system of W or X, wherein the one or more stabilizers is/are made of an elastomeric material, preferably an elastomer.
Z. The catheter system of any embodiment above, wherein the one or more stabilizers each have edge portions and a central portion, wherein the edge portions are affixed to the inner or outer surface of the second catheter shaft, and wherein the central portion is not affixed to the inner or outer surface of the second catheter shaft.
AA. The catheter system of any one of W-Z, wherein the second lumen of the second catheter has one or more inflation-fluid ports each being covered by the central portion of one stabilizer.
BB. The catheter system of any one of W-AA, having two or more stabilizers, wherein each stabilizer is made of a different elastomeric material, preferably having a different coefficient of friction, elasticity, deformability and/or compliance.
CC. The catheter system of any one of W-BB, wherein the elastomeric material of the central portion of the stabilizer is more flexible and/or has a greater elasticity than the elastomeric material of the edge portions of the stabilizers.
DD. A percutaneous transluminal angioplasty apparatus including:
   a support catheter; and
   a percutaneous transluminal angioplasty catheter that is inserted into the support catheter, the percutaneous transluminal angioplasty catheter having
      a distal end and a catheter balloon mounted to the distal end,
      an inflation lumen,
      a guide-wire lumen, and
   one or more hydraulically inflatable stabilizers,
   wherein the inflation lumen is in fluid communication with an internal volume of the catheter balloon and the one or more hydraulically inflatable stabilizers.
EE. The percutaneous transluminal angioplasty apparatus of DD, wherein the one or more stabilizers each includes:
   a band of stabilizer elastomeric material mounted to the surface of the percutaneous transluminal angioplasty catheter shaft; and
   one or more inflation-fluid ports below a central portion of the band of stabilizer elastomeric material.
FF. The percutaneous transluminal angioplasty apparatus of DD or EE, wherein a central portion of the band of stabilizer elastomeric material is not affixed to the outer surface of the percutaneous transluminal angioplasty catheter shaft;
   wherein two edge portions of the band of stabilizer elastomeric material are affixed to the outer surface of the percutaneous transluminal angioplasty catheter shaft, forming an expandable, annular inflation-fluid chamber between the central portion of the band of stabilizer elastomeric material and the underlying outer surface of the percutaneous transluminal angioplasty catheter shaft and one or more inflation-fluid ports.
GG. The percutaneous transluminal angioplasty apparatus of FF, wherein the inflation-fluid chamber is adapted to be inflated radially outward, compressing the stabilizer against the inner surface of the support-catheter shaft, by a pressurized inflation fluid, when the pressurized inflation fluid is introduced into the inflation lumen of the percutaneous transluminal angioplasty catheter and the pressurized inflation fluid flows through the inflation-fluid ports into the annular inflation-fluid chamber.
HH. The percutaneous transluminal angioplasty apparatus of any one of DD to GG, wherein the band of stabilizer elastomeric material is a portion of band of a portion of a stabilizer elastomeric material that forms two or more adjacent stabilizers along the percutaneous transluminal angioplasty catheter shaft, each overlying one or more inflation-fluid ports in the percutaneous transluminal angioplasty catheter shaft.
II. The percutaneous transluminal angioplasty apparatus of any one of DD to HH, wherein the percutaneous transluminal angioplasty catheter shaft includes two or more stabilizers, each including a band of stabilizer elastomeric material with an elasticity, deformability and/or compliance different from that of the band or bands of the other stabilizers and/or different from the material of the percutaneous transluminal angioplasty catheter balloon.
JJ. The percutaneous transluminal angioplasty apparatus of any one of A to P or DD to HH, wherein the elastomeric material of the stabilizers, preferably the elastomeric material of the central portion of the stabilizers, is more flexible and/or has a greater elasticity than the elastomeric material of the percutaneous transluminal angioplasty catheter balloon.
KK. The percutaneous transluminal angioplasty apparatus of any one of A to P or DD to JJ, wherein the support catheter has a shaft with a uniform circumference and/or diameter.

1. A catheter system comprising:
a first catheter having a first lumen surrounded by a first catheter shaft; and
a second catheter that is inserted into the first lumen of the first catheter, wherein,
the second catheter comprises a second lumen surrounded by a second catheter shaft and one or more hydraulically inflatable stabilizers being in fluid communication with the second lumen, and wherein the one or more stabilizers is/are adapted when being hydraulically inflated via the second lumen to form an inflation-fluid chamber being inflatable radially outward to compress the stabilizer against an inner surface of the first catheter shaft and fix the position of the second catheter within the first catheter and constrain the second catheter to a coaxial disposition with respect to the first catheter, wherein the first catheter comprises a support catheter or a guiding catheter and the second catheter comprises a percutaneous transluminal angioplasty (PTA) catheter.

2. The catheter system according to claim 1, wherein the PTA catheter comprises a balloon-length-adjustable PTA catheter.

3. The catheter system of claim 1, wherein a proximal portion of a balloon of the PTA catheter is recessed within the support catheter.

4. The catheter system of claim 1, wherein the one or more stabilizers each comprise edge portions and a central portion, wherein the edge portions are affixed to an inner or outer surface of the second catheter shaft, and wherein the central portion is not affixed to the inner or outer surface of the second catheter shaft.

5-7. (canceled)

8. A percutaneous transluminal angioplasty (PTA) apparatus comprising:
a support catheter; and
a percutaneous transluminal angioplasty (PTA) catheter within the support catheter, the percutaneous transluminal angioplasty catheter comprising,
a distal end and a catheter balloon mounted to the distal end,
an inflation lumen,
a guide-wire lumen, and
one or more hydraulically inflatable stabilizers,
wherein the inflation lumen is in fluid communication with an internal volume of the catheter balloon and the one or more hydraulically inflatable stabilizers.

9. A percutaneous transluminal angioplasty apparatus comprising:
a guide wire;
a support catheter; and
a percutaneous transluminal angioplasty (PTA) catheter within the support catheter, the percutaneous transluminal angioplasty catheter comprising,
a distal end,
a proximal end,
an outer shaft
a catheter balloon mounted to the distal end,
an inflation lumen in fluid communication with the internal volume of the catheter balloon,
a guide-wire lumen through which the guide wire passes, and
one or more stabilizers, that, when activated via the inflation lumen, fix the position of the percutaneous transluminal angioplasty catheter within the support catheter and constrain the percutaneous transluminal angioplasty catheter to a coaxial disposition with respect to the support catheter.

10. The PTA apparatus of claim 8, wherein the PTA catheter comprises a balloon-length-adjustable PTA catheter.

11. The PTA apparatus of claim 8, wherein a proximal portion of a balloon of the PTA catheter is recessed within the support catheter.

12. The PTA apparatus of claim 8, wherein the one or more stabilizers each comprises:
a band of stabilizer elastomeric material mounted to the surface of the percutaneous transluminal angioplasty catheter shaft; and
one or more inflation-fluid ports below a central portion of the band of stabilizer elastomeric material.

13. The PTA apparatus of claim 12, wherein a central portion of the band of stabilizer elastomeric material is not affixed to the outer surface of the PTA-catheter shaft;
wherein two edge portions of the band of stabilizer elastomeric material are affixed to the outer surface of the PTA-catheter shaft, forming an expandable, annular inflation-fluid chamber between the central portion of the band of stabilizer elastomeric material and an underlying outer surface of the PTA-catheter shaft and one or more inflation-fluid ports.

14. The PTA apparatus of claim 13, wherein the inflation-fluid chamber is adapted to be inflated radially outward, compressing the stabilizer against the inner surface of the support-catheter shaft when pressurized inflation fluid is introduced into the inflation lumen of the PTA catheter and the pressurized inflation fluid flows through the inflation-fluid ports into the annular inflation-fluid chamber.

15-17. (canceled)

18. The PTA apparatus of claim 12, wherein the band of stabilizer elastomeric material has an elasticity, deformability and/or compliance different from that of the PTA-catheter balloon so that the stabilizer is fully inflated at a different point in time than the point of time when the PTA-catheter balloon is fully inflated.

19. The PTA apparatus of claim 18, wherein the stabilizer is configured to be inflated prior to partial inflation of the PTA-catheter balloon.

20. The PTA apparatus of claim 18, wherein the stabilizer is configured to be inflated when the PTA-catheter balloon is partially inflated.

21. The PTA apparatus of claim 18, wherein the stabilizer is configured to be inflated when the PTA-catheter balloon is fully inflated.

22. The PTA apparatus of claim 18, wherein the stabilizer is configured to be inflated after the PTA-catheter balloon is fully inflated.

23. The PTA apparatus of claim 18, wherein the stabilizer is configured to be inflated together with a portion of the PTA-catheter balloon that is recessed within the support catheter, resulting in a combination of radial forces of the recessed PTA balloon portion and stabilizer, and applied to the support catheter, that counteract axial forces produced by balloon inflation, thereby fixing the position of the PTA catheter relative to the support catheter and constraining the PTA catheter to a coaxial disposition with respect to the support catheter.

24. The PTA apparatus of claim 18, wherein the stabilizer is configured to be inflated together with the PTA-catheter balloon recessed within the support catheter, resulting in a combination of radial forces of the recessed PTA balloon and stabilizer, and applied to the support catheter, that fix the position of the PTA catheter relative to the support catheter, thereby reducing or preventing strain, or forces, that are otherwise exerted, during push or pull operations of the PTA catheter.

25-28. (canceled)

29. The PTA apparatus of claim 9, wherein the PTA catheter comprises a balloon-length-adjustable PTA catheter.

30. The PTA apparatus of claim 9, wherein a proximal portion of a balloon of the PTA catheter is recessed within the support catheter.
